(19) United States
(12) Patent Application Publication
(10) Pub. No.: US 2019/0274526 A1
(43) Pub. Date: Sep. 12, 2019

(54) STEREOSCOPIC IMAGE PICKUP APPARATUS AND STEREOSCOPIC ENDOSCOPE

(71) Applicant: OLYMPUS CORPORATION, Tokyo (JP)

(72) Inventors: Kazuhiro MATSUMOTO, Tokyo (JP); Takeshi SAITO, Tokyo (JP)

(73) Assignee: OLYMPUS CORPORATION, Tokyo (JP)

(21) Appl. No.: 16/426,500

(22) Filed: May 30, 2019

Related U.S. Application Data
(63) Continuation of application No. PCT/JP2016/086310, filed on Dec. 7, 2016.

(51) Int. Cl. A61B 1/00; A61B 1/06; A61B 1/04; G02B 23/24; H04N 5/225

(52) U.S. Cl. CPC A61B 1/00193; A61B 1/00096; A61B 1/0676; H04N 2005/2255; G02B 23/2415; H04N 5/225; A61B 1/04

(57) ABSTRACT

A stereoscopic image pickup apparatus includes: a fixed barrel; a moving frame that is positioned in the fixed barrel so as to be advanceable and retractable and holds a plurality of moving lenses in a parallelly provided manner; and an actuator positioned in a space formed between the fixed barrel and the moving frame in a direction orthogonal to a line connecting photographing optical axes of the plurality of moving lenses and configured to drive the moving frame along the photographing optical axes.

### CROSS REFERENCE TO RELATED APPLICATION

This application is a continuation application of PCT/JP2016/086310 filed on Dec. 7, 2016, the entire contents of which are incorporated herein by this reference.

### BACKGROUND OF THE INVENTION

### 1. Field of the Invention

The present invention relates to a stereoscopic image pickup apparatus and a stereoscopic endoscope capable of displaying an object and sterically observing the object.

### 2. Description of the Related Art

In recent years, an endoscope apparatus capable of observing a site to be examined that cannot be directly checked with eyes by inserting an elongated insertion portion into a body cavity or the like, has been widely used.

In normal endoscope apparatuses, the site to be examined can only be seen as a plane surface without perspective. Therefore, normal endoscope apparatuses have a problem that, for example, a minute irregularity and the like on a wall surface of the body cavity are difficult to observe, and diagnosis and various treatment cannot be easily performed by the endoscope observation.

Thus, a stereoscopic endoscope apparatus in which a part to be observed can be stereoscopically seen by providing a plurality of observation optical systems in parallel and arranging the observation optical systems so as to obtain a parallax by setting the convergence angles formed by photographing optical axes of the optical systems is known.

As stereoscopic endoscopes as above, for example, a stereoscopic endoscope as a stereoscopic endoscope disclosed in Japanese Patent Application Laid-Open Publication No. 2014-140594 is known. The stereoscopic endoscope of the related art includes a feature in which the visual direction of the endoscope can be changed by bending photographing optical axes by mirrors and causing the bending photographing optical axes to form an image at the center side of the endoscope and by causing image sensors to move in the front-rear direction of the endoscope.

### SUMMARY OF THE INVENTION

A stereoscopic image pickup apparatus according to one aspect of the present invention includes: a fixed barrel; a moving frame that is positioned in the fixed barrel so as to be advanceable and retractable and holds a plurality of moving lenses in a parallelly provided manner; and an actuator positioned in a space formed between the fixed barrel and the moving frame in a direction orthogonal to a line connecting photographing optical axes of the plurality of moving lenses and configured to drive the moving frame along the photographing optical axes.

A stereoscopic endoscope according to one aspect of the present invention includes an insertion portion having a distal end portion in which a stereoscopic image pickup apparatus is positioned, the stereoscopic image pickup apparatus including: a fixed barrel; a moving frame that is positioned in the fixed barrel so as to be advanceable and retractable and holds a plurality of moving lenses in a parallelly provided manner; and an actuator positioned in a space formed between the fixed barrel and the moving frame in a direction orthogonal to a line connecting photographing optical axes of the plurality of moving lenses and configured to drive the moving frame along the photographing optical axes.

### BRIEF DESCRIPTION OF THE DRAWINGS

FIG. 1 is a perspective view illustrating the configuration of an endoscope apparatus that is a stereoscopic endoscope;

FIG. 2 is a schematic view illustrating a distal end portion of an insertion portion;

FIG. 3 is a perspective view illustrating the configuration of a moving lens unit in a fixed barrel;

FIG. 4 is a plan view illustrating the configuration of the moving lens unit in the fixed barrel;

FIG. 5 is a top view illustrating the configuration of the moving lens unit;

FIG. 6 is a perspective view illustrating a first example of the arrangement of permanent magnets in the moving lens unit;

FIG. 7 is a perspective view illustrating a second example of the arrangement of the permanent magnets in the moving lens unit;

FIG. 8 is a perspective view illustrating a third example of the arrangement of the permanent magnets in the moving lens unit; and

FIG. 9 is a plan view illustrating another example for rectilinearly guiding a moving frame.

### DETAILED DESCRIPTION OF THE PREFERRED EMBODIMENT(S)

An embodiment of the present invention is described below with reference to the drawings.

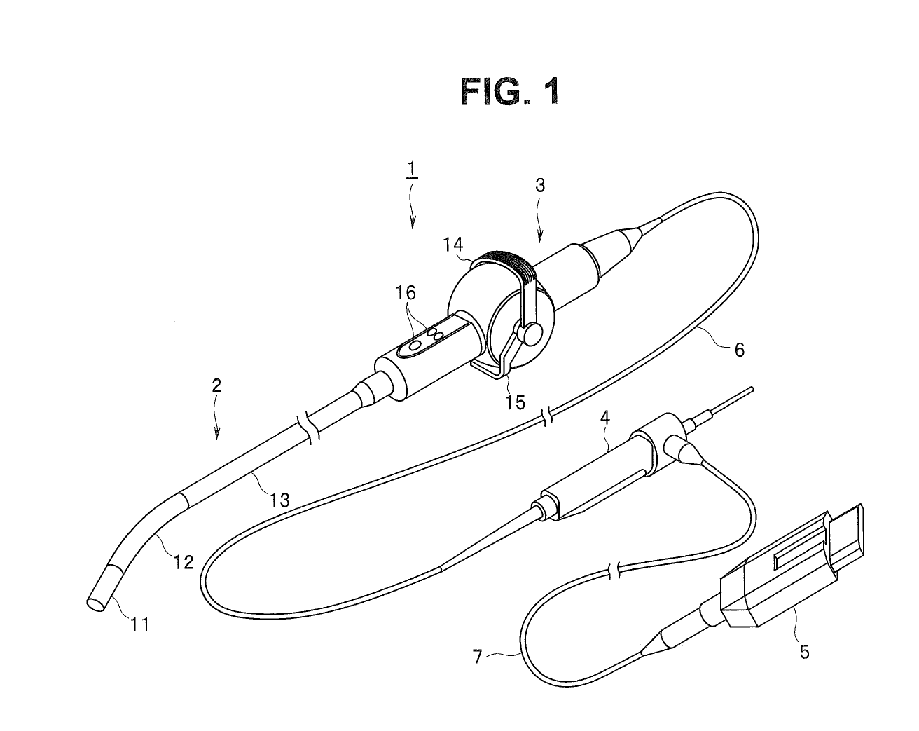

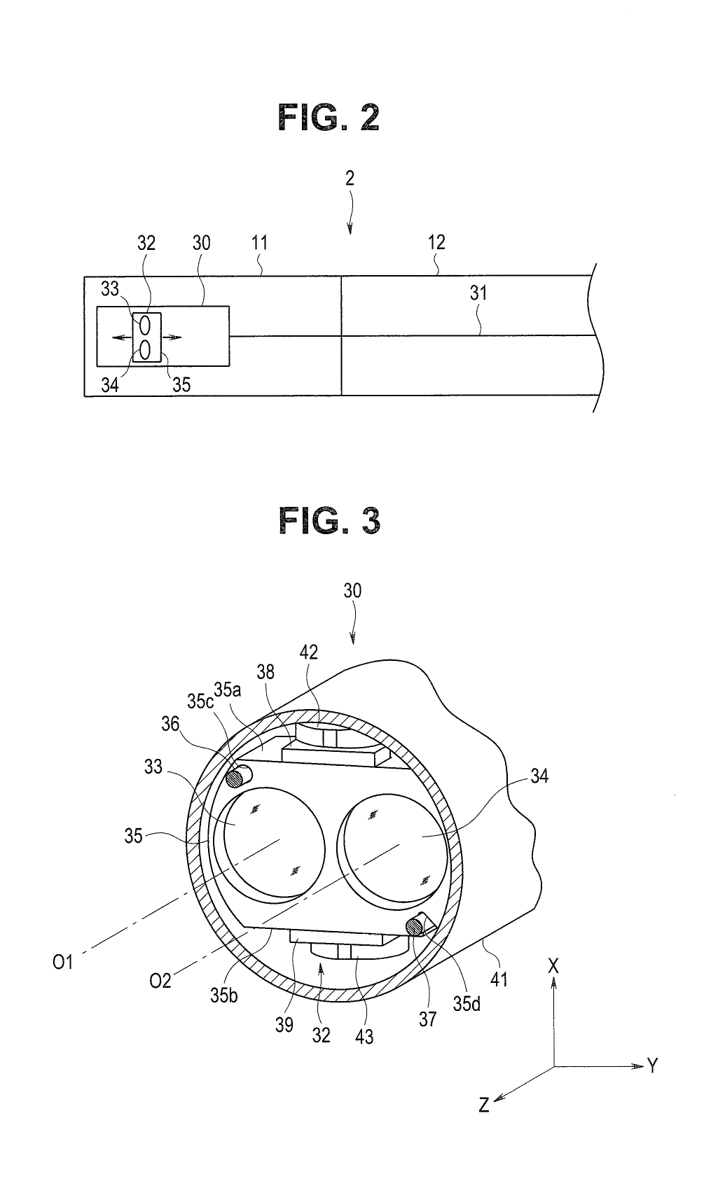

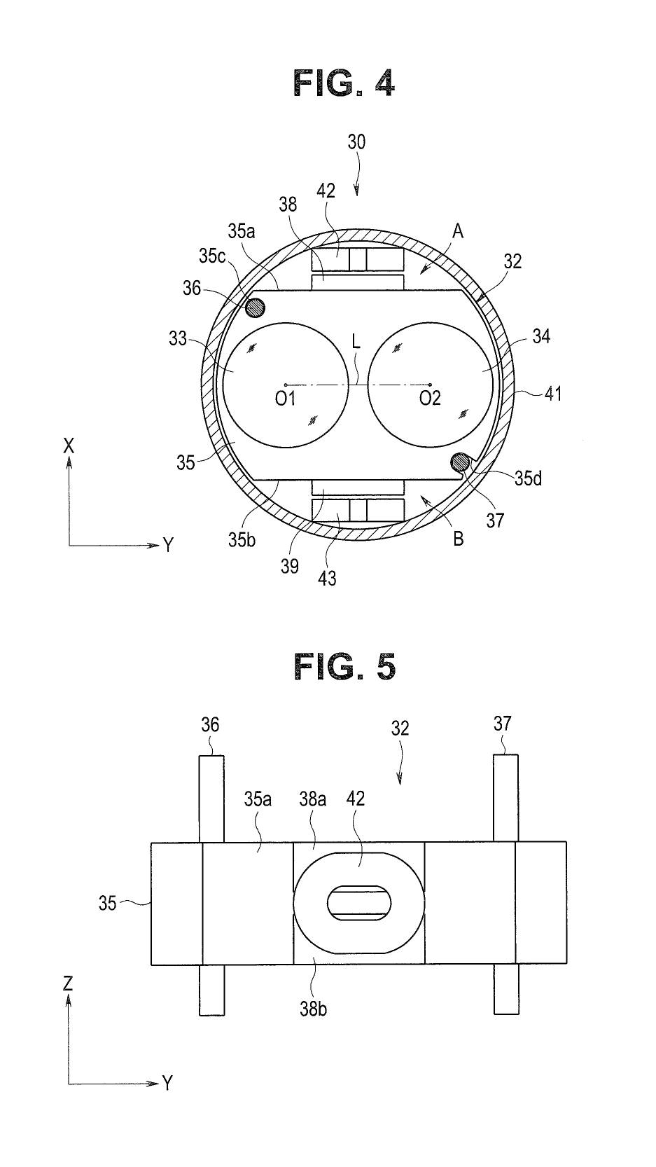

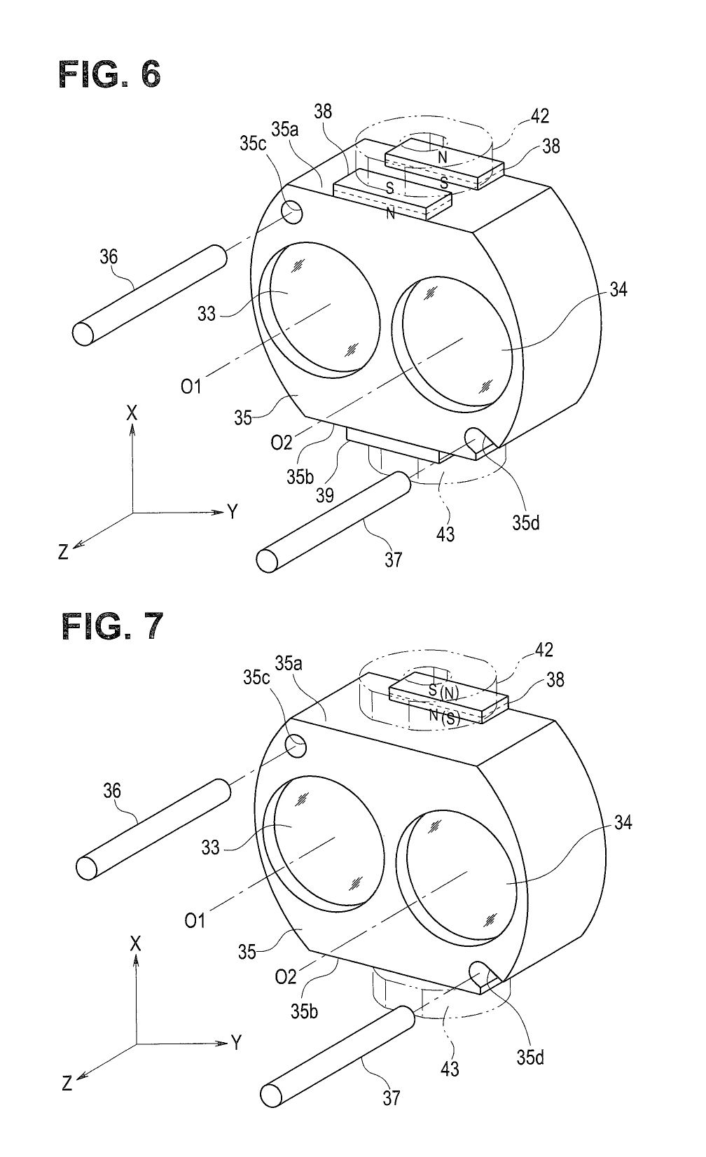

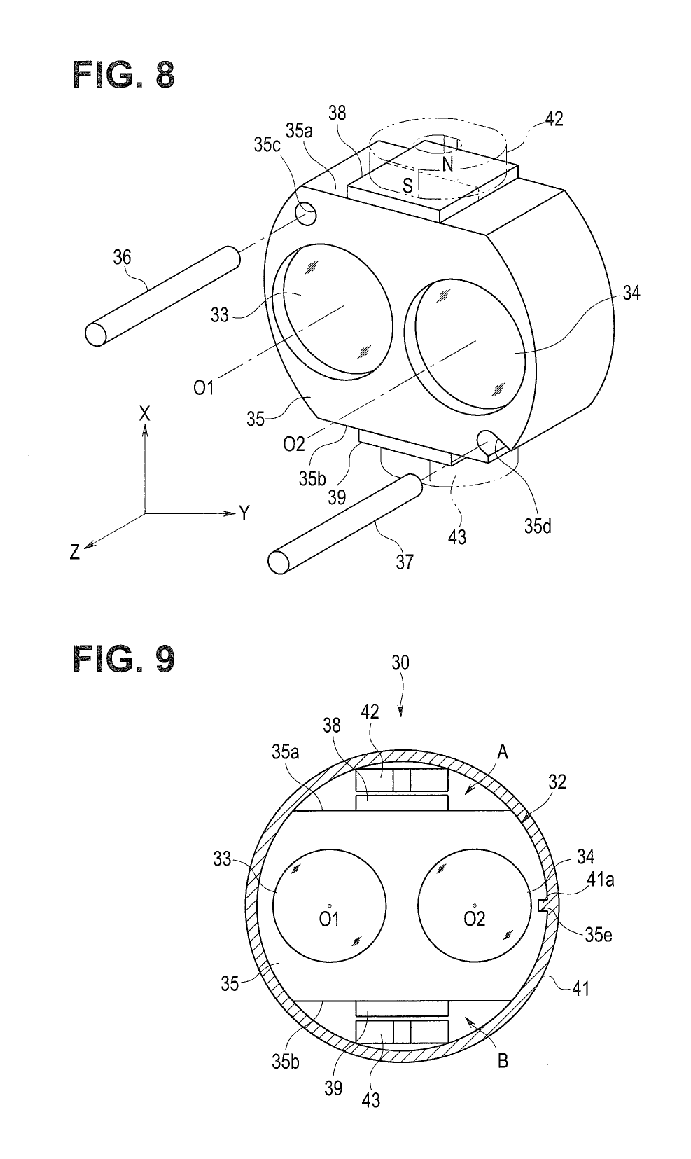

FIG. 1 is a perspective view illustrating the configuration of an endoscope apparatus that is a stereoscopic endoscope, FIG. 2 is a schematic view illustrating a distal end portion of an insertion portion, FIG. 3 is a perspective view illustrating the configuration of a moving lens unit in a fixed barrel, FIG. 4 is a plan view illustrating the configuration of the moving lens unit in the fixed barrel, FIG. 5 is a top view illustrating the configuration of the moving lens unit, FIG. 6 is a perspective view illustrating a first example of the arrangement of permanent magnets in the moving lens unit, FIG. 7 is a perspective view illustrating a second example of the arrangement of the permanent magnets in the moving lens unit, FIG. 8 is a perspective view illustrating a third example of the arrangement of the permanent magnets in the moving lens unit, and FIG. 9 is a plan view illustrating another example for rectilinearly guiding a moving frame.

Note that, in each of the drawings used in the description below, some scales are different for each component in order to cause each component to have a size that is recognizable on the drawings. In addition, the present invention is not limited to the quantity of the components, the shapes of the components, the ratio of the sizes of the components, and the relative positional relationship of the respective components illustrated in the drawings.

As illustrated in FIG. 1, an endoscope apparatus 1 serving as a stereoscopic endoscope is mainly configured by including a long insertion portion 2, an operation portion 3 provided so as to be connected to a proximal end of the insertion portion 2, a light guide connector 4 connected to a light source device (not shown), and a video connector 5 connected to a video system center (not shown).

Note that, in the endoscope apparatus 1, the operation portion 3 and the light guide connector 4 are connected to each other via a flexible cable 6, and the light guide connector 4 and the video connector 5 are connected to each other via a communication cable 7.

In the insertion portion 2, a distal end portion 11 mainly formed by a rigid member such as stainless steel and hard resin, a bending portion 12, and a rigid tube 13 that is a metal tube mainly made of stainless steel and the like are provided in a connected manner in the stated order from the distal end side. The insertion portion 2 is a portion inserted in the body, and various cables for communication and driving, a light guide (not shown) that transmits illumination light, and the like are built in the insertion portion 2.

The operation portion 3 includes angle levers 14 and 15 for remotely operating the bending portion 12, and various switches 16 for operating the light source device, the video system center, and the like. The angle levers 14 and 15 are bending operation means capable of operating the bending portion 12 of the insertion portion 2 in four directions, that is, up, down, left, and right directions. Note that the endoscope apparatus 1 of the present embodiment is a rigid endoscope apparatus in which a large part of the insertion portion 2 other than the bending portion 12 is rigid.

Next, with reference to FIG. 2, a stereoscopic image pickup apparatus (hereinafter abbreviated to an image pickup apparatus) 30 positioned on the distal end portion 11 of the insertion portion 2 is described.

As illustrated in FIG. 2, the image pickup apparatus 30 is positioned in the distal end portion 11, and a composite cable 31 obtained by bundling various cables for communication and driving is provided behind the image pickup apparatus 30 in an extending manner. The composite cable 31 is arranged in the insertion portion 2 in an inserted manner, and is electrically connected to the video connector 5 from the operation portion 3 via the flexible cable 6 and the communication cable 7.

In the image pickup apparatus 30, one or two image pickup devices (not shown) are positioned, and the image pickup apparatus 30 includes a circuit board (not shown) to which the image pickup device is electrically connected. Note that the image pickup device is an extremely compact electronic part. In the image pickup device, a plurality of elements that output electric signals in accordance with incident light at a predetermined timing are arranged on a planar light receiving portion. For example, types generally referred to as CCD (charge-coupled device) and CMOS (complementary metal oxide semiconductor) sensors or other various types are applied to the image pickup device.

Further, an image pickup signal that is photoelectrically converted by the one or two image pickup devices is generated into a video signal by the circuit board and is outputted. That is, in the present embodiment, an optical image (endoscope image) picked up by the one or two image pickup devices is transmitted to the video connector 5 as a video signal.

Note that, although the endoscope apparatus 1 of the present embodiment is a so-called 3D endoscope that can cause an image of an object to be a stereoscopic image, the principle for generating the stereoscopic image and the like are well known, and hence the description of the principle and the like is omitted.

In the image pickup apparatus 30, a plurality of objective optical systems configuring twin lenses for acquiring a stereoscopic image are positioned. Further, in the image pickup apparatus 30, out of the plurality of objective optical systems, a moving lens unit 32 including a moving frame 35 that holds two moving lenses 33 and 34 is positioned herein. Note that the number of the moving lenses 33 and 34 held by the moving frame is not limited to two.

As illustrated in FIG. 3 and FIG. 4, in a fixed barrel 41 of the image pickup apparatus 30, the moving lens unit 32 is positioned so as to be advanceable and retractable in a Z-axis direction in FIG. 3 along photographing optical axes O1 and O2 of the two moving lenses 33 and 34. Note that the fixed barrel 41 is a cylindrical member herein.

As described above, in the moving lens unit 32, the moving frame 35 holds the two moving lenses 33 and 34 that are the objective optical systems in a parallelly provided manner. The moving frame 35 is positioned in the fixed barrel 41 so as to be advanceable and retractable.

Two plane surface portions 35a and 35b along a Y-axis in FIG. 4 that are parallel with a line L connecting the centers of the two held moving lenses 33 and 34 through which the respective photographing optical axes O1 and O2 pass are formed on the top and the bottom of the moving frame 35. Permanent magnets 38 and 39 are respectively positioned on the two plane surface portions 35a and 35b in accordance with a predetermined magnetized direction.

That is, the plane surface portions 35a and 35b are formed on the top and the bottom of the moving frame 35 positioned in the cylindrical fixed barrel 41, and hence spaces A and B (see FIG. 4) are formed between the plane surface portions 35a and 35b and an inner circumferential surface of the fixed barrel 41. Further, the fixed barrel 41 forms the spaces A and B with the moving frame 35, and the spaces A and B serve as spaces for installing the permanent magnets 38 and 39 on the moving frame 35.

In addition, coils 42 and 43 are positioned on the fixed barrel 41 sides in the spaces A and B, respectively. The two coils 42 and 43 are wound around an axis parallel with an X-axis in FIG. 4 orthogonal to the line L connecting the centers of the two moving lenses 33 and 34 through which the respective photographing optical axes O1 and O2 pass.

Note that the coils 42 and 43 are respectively fixed to upper and lower inner surfaces of the fixed barrel 41 by an adhesive agent and the like, and are electrically connected to an electric cable in the composite cable 31. In each of the coils 42 and 43, the generated magnetic field direction is switched when the electric conduction direction is switched.

Incidentally, as illustrated in FIG. 5, the moving frame 35 is rectilinearly guided at the time of advance and retraction without revolving in the fixed barrel 41 by two shafts 36 and 37 serving as guiding portions. The two shafts 36 and 37 rectilinearly guide the moving frame 35 in a diagonal direction of the moving frame 35.

As illustrated in FIG. 6, in the moving frame 35, a hole portion 35c in which the shaft 36 is inserted in an engaged manner is formed on the plane surface portion 35a side on the upper part on the sheet of FIG. 6, and a U-shaped groove 35d in which the shaft 37 is inserted in an engaged manner is formed on the plane surface portion 35b side on the lower side on the sheet of FIG. 6. Although not shown, the two shafts 36 and 37 are fixed to the fixed barrel 41 in a state of being inserted in the moving frame 35 in an engaged manner.

Stoppers (not shown) that define the advance and retraction range of the moving frame 35 are provided on the two shafts 36 and 37. Note that the stoppers that define the advance and retraction range of the moving frame 35 may be provided by simply providing protrusions that abut against the front and the rear of the moving frame 35 on the fixed barrel 41.

Incidentally, as illustrated in FIG. 6, for example, when two of the permanent magnets 38 and 39 are provided on each of the plane surface portions 35a and 35b of the moving frame 35, S and N poles are magnetized in a direction orthogonal to the photographing optical axes O1 and O2 and S and N polarities on the front and the rear are fixed in an opposite state.

In addition, as illustrated in FIG. 7, for example, when only one of the permanent magnets 38 and 39 is provided on each of the plane surface portions 35a and 35b of the moving frame 35, the S and N poles are magnetized in a direction orthogonal to the photographing optical axes O1 and O2 and are fixed on one side in the front-rear direction of the moving frame 35 so as to be shifted to one of the front and the rear of the coils 42 and 43 (the side is illustrated to be the rear side of the moving frame 35 in FIG. 7 but also may be the front side).

Further, as illustrated in FIG. 8, for example, when only one of the permanent magnets 38 and 39 is provided on each of the plane surface portions 35a and 35b of the moving frame 35, the S and N poles are magnetized in a direction along the photographing optical axes O1 and O2 and are fixed to the moving frame 35 so as to evenly extend over the front and the rear of the coils 42 and 43.

As described above, by the permanent magnets 38 and 39 fixed to the moving frame 35 and the coils 42 and 43 that switch a gravitational force and a repulsive force with respect to the permanent magnets 38 and 39, a VCM is configured and an actuator serving as a driving source that causes the moving lens unit 32 to advance and retract is configured.

Note that, in the present embodiment, a configuration in which the moving lens unit 32 is moved to advance and retract by the two coils 42 and 43 is exemplified, but it is also possible to provide one coil and a permanent magnet that forms a pair with the coil on only one of the plane surface portions 35a and 35b of the moving frame 35.

In addition, the configuration of the guiding portion that rectilinearly guides the advance and the retraction of the moving frame 35 may be a configuration of a guiding portion that rectilinearly guides the moving frame 35 by causing the moving frame 35 to be radially fitted in the inner circumferential surface of the fixed barrel 41, forming a key groove 35e along an advancing/retracting direction in a part of the moving frame 35, and forming a protrusion portion 41a engaged with the key groove 35e on the fixed barrel 41 as illustrated in FIG. 9, for example, and not by the two shafts 36 and 37.

In the embodiment described above, a configuration in which the moving frame 35 is caused to advance and retract in the fixed barrel 41 of the image pickup apparatus 30 is exemplified, but the fixed barrel 41 may be an exterior frame of the distal end portion 11 of the insertion portion 2 as long as the configuration of the stereoscopic endoscope does not include illumination, a treatment instrument channel, and the like.

Further, in the present embodiment, the rigid endoscope is exemplified, but the present invention is not limited to the rigid endoscope, and is a feature that can also be applied to a flexible endoscope and an industrial endoscope.

In the endoscope apparatus 1 that is a stereoscopic endoscope of the present embodiment described above, the actuator serving as a VCM is arranged in a direction orthogonal to the direction in which the moving lenses 33 and 34 that are two objective optical systems as the twin lenses are parallelly provided.

Further, in the endoscope apparatus 1, by the configuration in which the coils 42 and 43 of the VCM are wound around in the direction in which the direction orthogonal to the line L connecting the photographing optical axes O1 and O2 of the two moving lenses 33 and 34 is the axis, the thickness of the coils 42 and 43 becomes equivalent to the number of turns of the coil wire.

Therefore, the upsizing in the parallelly provided direction of the two moving lenses 33 and 34 (the extending direction of the line L) can be prevented, the actuator that is a VCM can fit into the spaces A and B that are gaps between the inner circumferential surface of the fixed barrel 41 of the image pickup apparatus 30 and the moving frame 35 that holds the two moving lenses 33 and 34, and the outer diameter of the image pickup apparatus 30 can be prevented from increasing. That is, the image pickup apparatus 30 can have a configuration including a zoom function or a focus function that prevents the upsizing in the direction orthogonal to the photographing optical axes O1 and O2.

As a result, in the endoscope apparatus 1, the upsizing can also be prevented because the outer diameter of the distal end portion 11 of the insertion portion 2 on which the image pickup apparatus 30 is positioned does not increase.

The feature configuration and the effects of the image pickup apparatus and the stereoscopic endoscope of the present embodiment include the following.

The stereoscopic endoscope of the present embodiment includes the twin lenses, the moving frame that holds the twin lenses so as to be advanceable and retractable along photographing optical axis, and the actuator serving as the driving source of the moving frame, in which the actuator is arranged so as to be separated in a direction orthogonal to the line connecting the photographing optical axes of the twin lenses.

Therefore, the actuator serving as the VCM is not arranged in the extending direction of the line connecting the photographing optical axes of the twin lenses, and hence does not stick out in the parallelly provided direction of the twin lenses. Therefore, the upsizing of the image pickup apparatus can be prevented. Accordingly, the outer diameter of the distal end portion of the insertion portion of the endoscope apparatus also does not increase.

In addition, by causing the actuator to be a VCM including the coil and the magnet, the degree of freedom in the arrangement increases as compared to a rotary motor, and the transmission loss in the power is small. Therefore, a configuration in which the image pickup apparatus 30 can be downsized and lightened, reduced in power consumption, and smoothly driven, for example, can be obtained.

The number of the actuator that is a VCM of the image pickup apparatus 30 only needs to be determined to be one or a plurality in consideration of the needed driving force and the empty space on the inside.

The VCM is a moving magnet type in which the coil is arranged on the fixed barrel on the fixed side and the permanent magnet is arranged on the moving frame on the movable side and does not need wiring on the movable side, and hence can have a configuration with excellent assembling property.

By winding the coils of the VCM around the axis in the direction orthogonal to the line connecting the photographing optical axes of the twin lenses, the coils can be arranged without expanding the diameter of the image pickup apparatus and the distal end portion of the endoscope apparatus as compared to when the coils are wound in the axis direction along the photographing optical axes.

Note that the VCM is set so as to generate a driving force in the advance and retraction direction of the moving lens by the Lorentz force of the coils and the permanent magnets, but the magnetic field from the permanent magnet describes an arc when leaving the magnet and returning to the magnet. Therefore, the VCM only needs to be arranged in a position on the plane surface portion of the moving frame that can provide the necessary magnetic field direction, and the degree of freedom in the arrangement is high.

The mechanism that holds the moving frame that holds the twin lenses to be advanceable and retractable in the photographing optical axis direction performs rectilinear guidance by the two shafts, while positioning the rotation center by one shaft and restricting the rotation by the other shaft.

In the configuration of rectilinearly guiding and holding the moving frame by the two shafts as above, the position is highly accurate because the correction and the assembly adjustment of the parts can be performed, and driving can be performed with small amount of loss due to sliding resistance because friction is small and viscous resistance is small even when grease is applied.

In the mechanism that holds the moving frame that holds the twin lenses to be advanceable and retractable in the photographing optical axis direction, the moving frame that moves and the fixed barrel that does not move are radially fitted together, and the protrusion that is a key for rotation restriction and the key groove are provided.

By the radial fitting as above, the load is distributed to a large area and strong force is not applied to one portion even when external force is applied to the moving frame that moves, and hence the stiffness can be increased.

The invention described in the embodiment above is not limited to the abovementioned embodiment, and other various modifications can be made without departing from the gist of the invention on an implementation stage. Further, in the abovementioned embodiment, various stages of the invention are included, and various inventions can be extracted by appropriate combinations of the plurality of disclosed components.

For example, even when some components are deleted from all the components described in the embodiment, the configuration in which the components are deleted can be extracted as the invention when the described problem can be solved, and the described effect can be obtained.

What is claimed is:

1. A stereoscopic image pickup apparatus, comprising:
a fixed barrel;
a moving frame that is positioned in the fixed barrel so as to be advanceable and retractable and holds a plurality of moving lenses in a parallelly provided manner; and
an actuator positioned in a space formed between the fixed barrel and the moving frame in a direction orthogonal to a line connecting photographing optical axes of the plurality of moving lenses and configured to drive the moving frame along the photographing optical axes.

2. The stereoscopic image pickup apparatus according to claim 1, wherein the moving frame comprises a plane surface portion that forms the space, the plane surface portion being parallel with the line connecting the photographing optical axes of the plurality of moving lenses.

3. The stereoscopic image pickup apparatus according to claim 1, wherein the actuator is a voice coil motor comprising a permanent magnet and a coil.

4. The stereoscopic image pickup apparatus according to claim 3, wherein the permanent magnet is positioned on the moving frame, and the coil is fixed to the fixed barrel.

5. The stereoscopic image pickup apparatus according to claim 1, further comprising a guiding portion configured to rectilinearly guide the moving frame.

6. A stereoscopic endoscope comprising an insertion portion having a distal end portion in which the stereoscopic image pickup apparatus according to claim 1 is positioned.
